(19) United States
(12) Patent Application Publication
(10) Pub. No.: US 2011/0144687 A1
(43) Pub. Date: Jun. 16, 2011

(54) Lateral Based Retractor System

(76) Inventor: Jeffrey Kleiner, Aurora, CO (US)

(21) Appl. No.: 12/965,654

(22) Filed: Dec. 10, 2010

Related U.S. Application Data
(60) Provisional application No. 61/262,075, filed on Dec. 10, 2009.
(60) Provisional application No. 61/323,984, filed on Apr. 14, 2010.

(51) Int. Cl. A61M 29/00

(52) U.S. Cl. 606/192

(57) ABSTRACT

An improved system for lateral based minimally invasive surgery is disclosed. More specifically, devices for use in spinal surgical procedures are disclosed that include asymmetric cross-sections which offer a reduced risk of impingement upon nerves and other tissues and the post-operative complications associated with such. Additional features including anchoring means, fiber optic lighting and image sensing means may be provided to improve visibility and surgical navigation during procedures.

The present application is a non-provisional patent application which claims the benefit of priority from commonly owned and co-pending U.S. Provisional Application No. 61/262,075, filed Dec. 10, 2010, and 61/323,984, filed Apr. 14, 2010, the entire contents of which are hereby expressly incorporated by reference in this disclosure as if set forth fully herein.

### FIELD OF THE INVENTION

The present disclosure relates generally to a method and apparatus for use in spinal surgery. More specifically, the present disclosure relates to instruments and methods of use for lateral based spinal surgery that allow for sufficient access to and illumination of a surgical area with reduced risk of impinging upon or damaging surrounding bone, tissue, etc. U.S. Provisional Patent Application No. 61/262,075, filed Dec. 10, 2009, is incorporated by reference herein in its entirety.

### BACKGROUND OF THE INVENTION

Conventional surgical procedures and methods typically require cutting of back muscles, removal of bone, and retraction of other natural elements. Lateral based spinal surgery is a known alternative to conventional surgical procedures, and is generally referred to as a “minimally-invasive” procedure. Lateral based procedures offer the advantages of shorter recovery times, reduced blood loss, reduced post-operative complications, and shorter operating times than conventional procedures and methods. For example, one surgical approach for spinal fusion using a minimally invasive technique is known as “lumbar interbody fusion,” or LIF for short. Other known examples of lateral based approaches include the Nuvasive XLIF procedure and Medtronic D-LIF System. However, these systems and methods have problems, including, but not limited to, limited visualization and lighting in the surgical area, increased risk of impinging upon nerves such as the femoral nerve and psoas nerve, and the risk of devices and/or instruments becoming dislodged during the various procedures. These problems, alone or in combination, may result in post-operative pain and discomfort experienced by patients of lateral based spinal surgery. In some instances, these problems result in additional surgeries, further complicating the likelihood of recovery and successful fusion.

Various retractor devices and surgical access systems are known in the art to facilitate minimally invasive surgical procedures while allowing for a sufficiently large surgical area. Retractors are useful for gradually dilating the area of an incision or surgical opening in order to form a desired amount of space within which various procedures may be conducted. These devices may include a series of tools which, when consecutively inserted, serve to gradually expand an area. Similarly, retractors may take the form of a single device that may be inserted into a work area and expanded at the direction of a user, thus allowing for the creation and maintaining of a surgical work space.

There has been a long-felt but unmet need for a lateral based refractor system for use in surgery that provides both adequate workspace and sufficient visibility while reducing the risk of impingement and damage to surrounding nerves and tissue. The following disclosure describes a lateral based retractor system that employs a novel combination of features that address all of these long felt needs.

### SUMMARY OF THE INVENTION

The present invention improves upon prior art minimally invasive surgical apparatus and methods by addressing these and other problems in the art. For example, U.S. Patent Application Publication No. 2007/0213596 to Hamada, which is hereby incorporated by reference in its entirety, discloses an apparatus for a retractor which is capable of forming and maintaining a surgical area. Hamada discloses a generally cylindrical tubular apparatus for accessing the desired surgical area. Hamada does not teach novel aspects of the present invention, namely the ability to expand a surgical area without impinging upon nerves and surrounding tissue.

U.S. Patent Application Publication No. 2008/0009929 to Harris et al., which is hereby incorporated by reference in its entirety, discloses a technique for percutaneously introducing a stimulation lead into a surgical area. Harris et al. discloses a device with a substantially conical distal end that may be inserted around a guide wire to widen the path through an epidural region of a patient. Harris et al. fails to address the risk of impinging upon nerves and other tissue and therefore does not teach novel aspects of the present invention.

U.S. Patent Application Publication No. 2005/0149035 to Pimenta et al., which is hereby incorporated by reference in its entirety, discloses a surgical access system that may be used in lateral based transpsoas procedures. Pimenta et al. discloses, for example, a device that comprises a plurality of blades which may be inserted into a patient and selectively expanded away from each other in order to form an opening corridor for a surgical target site. Pimenta et al. fails to disclose devices and methods as taught by the present invention, whereby a series of devices are provided to gradually form a pathway for a surgical target area but maintain separation from another area with reduced risk of impinging upon nerves and surrounding tissue.

U.S. Patent Application Publication No. 2010/0036226 to Marino et al., which is hereby incorporated by reference in its entirety, discloses an expandable tip cannula system with an expandable tip and nerve sensing electrodes disposed therein. Various features of Marino et al. may be incorporated into embodiments of the present invention. For example, in one embodiment, the present invention comprises methods and systems for accessing a surgical target site comprising the use of one or more nerve stimulation electrodes and at least one expandable member provided.

U.S. Patent Application Publication No. 2010/0036384 to Gorek et al., which is hereby incorporated by reference in its entirety, discloses systems and methods for determining a desired trajectory and/or monitoring the trajectory of surgical instruments.

Various disclosed methods and features of Gorek et al. may be utilized in various embodiments of the present invention. For example, although Gorek et al. is generally directed to ensuring the proper placement of pedicle screws during fixation procedures, various features of Gorek et al. may be utilized to guide features of the present invention in transpsoas related procedures.

U.S. Patent Application Publication No. 2010/0036442 to Lauryssen et al., which is hereby incorporated by reference in its entirety, discloses various tools and devices for use in spinal surgery. Lauryssen et al. discloses a spinal guide device with a guide member having a positioning element adapted to engage a portion of a vertebra and a retractor guide adapted to receive and guide retractors. Various disclosed devices and features of Lauryssen et al. may be incorporated into embodiments of the present invention.

U.S. Patent Application Publication No. 2010/0010494 to Quirno, which is hereby incorporated by reference in its entirety, discloses a method and device for determining an optimal size of an intervertebral implant to be inserted within an intervertebral space in a patient.

According to varying embodiments disclosed herein, the present invention contemplates a group of surgical tools and method of using the same in lateral based spinal surgery and other minimally invasive or percutaneous surgical procedures. The invention comprises, according to one embodiment, a series of cannulas and dilators, including at least one pilot cannula and at least one dilator. In one embodiment, one or more sources of light are provided at a distal end of either or both the pilot cannula and dilator. Additionally, according to one embodiment, anchoring means are also provided to prevent or inhibit undesired movement once the various apparatus of the present invention are positioned in their desired location(s).

In another embodiment, the present invention comprises multiple cannulas for insertion in surgical procedures where the cannulas are shaped and oriented so as to avoid nerve injury or damage in a particular direction. For example, asymmetrical cannulas may be inserted into a surgical area in an orientation that allows for a sufficiently large surgical work area without the risk of impinging upon posterior nerves. By inserting progressively larger asymmetric cannulas according to this orientation described above, the visible and accessible surgical area may be dilated, but need not expand outwardly in a uniform radial fashion as do many currently known devices, specifically in the Nuvasive and Medtronic designs.

The present invention contemplates numerous asymmetric shapes which achieve the objective of progressively expanding a workspace or operative corridor in a non-circular manner so as to avoid or reduce the risk of nerve impingement. In a preferred embodiment, devices of the present invention comprise a “crescent moon” cross section. In an alternative embodiment, the present invention comprises cannulas of a half-circle cross section. One of skill in the art will recognize a wide array of different shaped cannulas which may accomplish objects of the present invention. It is to be expressly understood that the present invention is not limited to a cannula or cannulas of a particular cross-sectional shape. It is also to be expressly understood that, as used herein, cannula or cannulas refer generally to elongate objects for insertion into a patient and not limited to devices designed for performing any specific function. Cannulas of the present invention may be adapted for inserting or retracting medication or fluid, create an operative corridor for viewing and/or working inside a patient, etc.

Accordingly, in various embodiments, a system for accessing a surgical target site is provided, the system comprising a plurality of sequential dilators deliverable along a lateral, trans-psoas path to a targeted spinal site to create an operative or working corridor for various procedures. The system further comprises a pilot cannula or guide needle for use an initial tool or target identification features. A plurality of dilators having an asymmetric shape when viewed in cross-section are also provided, the dilators having a first end, a second, and a longitudinal length. Thus, the pilot cannula may be configured to introduce one or more dilators along a lateral, trans-psoas path toward the target site, thereby creating an operative corridor.

In various embodiments, a working cannula that is deliverable along the aforementioned lateral, trans-psoas path to a targeted spinal site may further be provided in order to maintain said operative corridor. Working cannulas of the present invention generally have an inner surface and a path for receiving surgical instruments and/or removal and inserting items from the workspace.

In a particular embodiment, the working cannula comprises a main body portion and an enclosed secondary housing. The enclosed secondary housing consists essentially of a parallel pathway to a main body portion of the working cannula. In one embodiment, the secondary pathway has a length substantially similar to the length of the main body portion and a longitudinal axis that is substantially parallel to the longitudinal axis of the main body portion. In various embodiments, the enclosed secondary housing comprises a fastener and a biasing element (e.g. coil spring) for biasing the fastener in a direction away from said surgical target site.

Furthermore, various methods for performing lateral based minimally invasive surgical procedures utilizing progressively larger asymmetric cannulas are provided. In one embodiment, a method is provided comprising providing a dilator system comprising a plurality of sequential dilators deliverable along a lateral, trans-psoas path to a targeted spinal site to create an operative corridor. Subsequently, a pilot cannula adapted for sensing the proximity of nerves is inserted into a patient at or near a desired operative or target site. A user or surgeon thereafter verifies that the pilot cannula is not within a predetermined distance to a nerve. A predetermined distance may be a variety of known acceptable distances as will be recognized by one of ordinary skill in the art. It will be recognized that such a distance may be largely dependent upon the size and shape of various tool to be subsequently inserted into the patient.

Where the verifying step indicates that the pilot cannula is not within the predetermined distance to a nerve, a first dilator is introduced, the first dilator having a first end, a second, and a longitudinal length therebetween. In various embodiments, the first dilator comprises an asymmetric cross-sectional shape. Subsequently, a second dilator is introduced about said first dilator, the second dilator having a first end, a second end, and a longitudinal length therebetween and having an asymmetric cross-sectional shape. One of skill in the art will recognize that additional dilators may be employed to achieve a desired surgical access point. Thereafter, a working cannula is introduced. The working cannula is deliverable along said lateral, trans-psoas path to a targeted spinal site and designed to maintain an operative corridor, and is secured at at least one of a distal end and a proximal end of said working cannula.

In various embodiments, this method further comprises determining whether the working cannula requires an extension cannula based on indicia provided on said working cannula. It is known that patients of various sizes require different length working cannula to adequately address lighting/visualization needs. Thus, one or more extension cannulas may be selected and secured to the working cannula. In this manner, a single working cannula potentially comprising valuable materials may be extended through the provision of a plurality of less complex or expensive features.

The present invention also contemplates the use of one or more embedded wire electrodes which operate to detect the proximity of features of the invention to nerves, as well as provide feedback to a user. U.S. Patent Application Publication No. 2009/0259108 to Miles et al., which is hereby incorporated by reference in its entirety, discloses a surgical access system and related methods including a plurality of retractor blades which may be equipped with one or more electrodes for use in detecting the existence and location of neural structures. Miles et al. discloses various methods for monitoring proximity to nerve structures, including observing visual twitches of muscles as well as various commercially available monitoring devices.

U.S. Pat. No. 7,819,801 to Miles et al., which discloses a surgical access system including a tissue distraction assembly and a tissue retraction assembly, is hereby incorporated by reference in its entirety.

Furthermore, one or more fibre optic devices may be utilized with the present invention to provide additional lighting or imaging of the surgical space. As one of the advantages of percutaneous procedures include minimal injury or incision in a patient, image sensing means serve to provide meaningful visual feedback on the surgical workspace without excessive removal or damage to a patient's tissue.

In certain embodiments, one aspect of the invention is directed to retrofitting existing instruments to provide the desired contoured shape of the bone contacting end of an instrument. For example, suitable disposable sleeves may be employed to slip over (or otherwise be associated therewith) exiting cannulas so that the tip end thereof can assume a desired shape, such as the asymmetrical shapes disclosed herein. Moreover, another aspect of the present invention relates to the use of different materials that have other desired attributes than may be associated with surgical steel. For example, the use of various composites, plastics, etc. such as PEEK, may be employed in either the manufacture of appropriate instruments in accordance with the present invention or in the provision of retrofit—adapters for existing instruments. As one will appreciate, the ability to provide disposable instruments that have the desired conformational and geometric shapes, lengths, flexibility, conformability, etc. to ensure that sensitive tissues are not adversely impacted is achievable by selectively using the desired shapes of cannulas made from various materials that are less prone to damage tissue than conventional steel instruments.

Still other aspects of the invention are directed to cannula instruments that have a patient contacting end that is adjustable to assume a predetermined conformation. Thus, in one embodiment, material forms the tip end that comes into contact with bone, tissue, and particularly near especially nerve tissue, with such cannula end material being malleable to an extent necessary for the surgeon to mold the end conformation such that it achieves desired avoidance of particular structures encountered in any particular surgery. Thus, if a bony outcropping, a nerve fiber, etc. is perceived by the surgeon, the cannula tip end can be adjusted to avoid undesired contact or interference with such tissues or structures. In particular embodiments, the ability to adjust the geometric parameters of the tip end is achieved by manipulation of the other end of the instrument. For example, providing a turnable component at the opposite end of the instrument, the shape of the other end of the instrument (i.e. the end inserted into the patient) can be adjusted to expand circumference, reduce circumference, render the opening more or less oblong, etc. In such a manner, it is possible to avoid having to remove the instrument or cannula from the patient's site to adjust the morphology of the instrument or cannula operating end, thus saving time, avoiding undesired reinsertion procedures, etc.

In yet another embodiment, the present invention includes at least one asymmetric dilator or cannula which is applied in a patient and the assembled working cannula and fibre optic attachment are placed over the dilator(s). In one embodiment, a combined unit is held together by a ridge, protrusion, extension, etc. disposed on a working cannula, which communicates with a complementary depression, recess, or void on a fibre optic cannula. A recession or slot may be formed within a portion of the cannulas to allow for elastic compression and/or expansion of at least a portion of the cannula and further lock or accommodate an additional cannula or device.

In one embodiment, the working cannula comprises a metal device of approximately 5 cm in length and is adapted for use in multiple procedures (i.e. may be subject to autoclaving and/or similar sanitization procedures). The working cannula may be modified to include an angled end corresponding to a non-perpendicular working angle of the disk space. In one embodiment, the end of the working cannula is angled at an angle between 8 and 15 degrees. In a more preferred embodiment, the end of the working cannula is angled at an angle between approximately 11 and 13 degrees. In a more preferred embodiment, the end of the working cannula is angled at approximately 12 degrees (i.e. 12 degrees with respect to a longitudinal axis of the cannula).

In one embodiment a cannula comprising fiber optic lighting and/or image sensing devices is provided. In one embodiment, the fiber optic cannula is comprised of a plastic, such as PEEK or other plastics suitable for use in surgical procedures as known to those skilled in the art. In one embodiment, a plurality of fiberoptic cannula is provided, whereby a cannula of appropriate size and length may be selected based upon patient size and dimensions. One of skill in the art will recognize that different combined cannula lengths (i.e. the total length of a docked or combined working and fiber optic cannula) may be desired based on different patient sizes and various other dimensional considerations and limitations. It is noted that as patient size increases, so too must the length of the cannula and/or combined unit in order for a proximal end of the device to reside external to the surgical workspace. However, particularly when dealing with more slender patients, it is undesirable to use a larger (i.e. longer) device as this typically results in the image sensing and/or light emitting device being positioned at an undesired distance away from the workspace. The present invention therefore contemplates providing a plurality of fiber optic cannulas that are varied in length and may be selected based upon patient size and other relevant factors. In one embodiment, these fiber optic cannula are designed for single patient use and thus disposable and/or recyclable.

In one embodiment, the fiber optic cannula further comprise one or more grooves, channels, pathways, indentations, canals, or passageways which serve as a guide for a screw, screw driver and/or various other devices, including, but not limited to, nerve stimulation devices as known to those of skill in the art. In one embodiment, these grooves or pathways comprise a fully enclosed channel which resides either outside or within an inner diameter of the fiber optic cannula. In an alternative embodiment, these grooves or pathways comprise a crescent shaped channel which substantially encloses devices to be housed, translated, or guided within the channels.

In one embodiment, a cannula with fiber optic imaging means or devices is provided. This fiber optic cannula may be provided in a variety of sizes and lengths, depending on patient size. In one embodiment, the fiber optic cannula has a length between about 2 cm and 12 cm. In a more preferred embodiment, the fiber optic cannula of the present invention has a length between 4 cm and 9 cm. Cannulas of different lengths may be provided at any number of intervals or increments. For example, fiber optic cannulas of the present invention may be provided at 4 cm, 5 cm, 6 cm, etc. and/or may be provided at uneven increments (e.g. at 4 cm, 4.8 cm, 5.3 cm, etc.).

In yet another embodiment, a working cannula is provided that comprises indicia along the length of the cannula useful for determining the appropriate size and/or length of fiber optic that should be employed. For example, the present invention contemplates a working cannula that comprises graduated markings along an exterior portion of the cannula, the graduated markings being concealed as the working cannula enters the skin edge. These graduated markings are proportional to or correspond with the length of the fiber optic cannula that should preferably be attached or docked to the working cannula. Thus, when a working cannula of the present invention is properly disposed within an operative site, the last visible graduated marking provides visual indicia to a user as to which fiber optic cannula should be employed to create optimal conditions (i.e. optimal lighting, focal length, and/or other visual and optical conditions). In one embodiment, visual indicia such as graduated markings comprise a color marking scheme (i.e. in addition to or in lieu of written markings) which correspond to the appropriate fiber optic device or cannula to be attached or docked with the working cannula.

In one embodiment, cannulas of the present invention comprise one or more compression gaps or slotted recess, which allows a portion of a cannula to expand and compress and attach to or frictionally engage a second cannula adapted for use with the first cannula. In a preferred embodiment, this compression gap is provided on at least a fiber optic cannula which is designed to be placed around and secure to a working cannula.

In another embodiment, cannulas of the present invention comprise ridges which are adapted to aid in docking or communicating with additional cannulas. In one embodiment, these ridges comprise annular protrusions around an inner circumference of a cannula which are capable of communication with either annular indentations or protrusions disposed on an outer portion of another cannula. Thus, docking and un-docking of multiple cannulas may be accomplished by the application of force, which results in positioning a ridge over or into corresponding ridges or recessions of another cannula.

In one embodiment, at least one cannula (e.g. a working cannula) of the present invention comprises a spring-loaded screw which may be anchored into a patient for the purposes of securing devices of the present invention. In one embodiment, a screw is disposed within a portion of the present invention, such as a pathway that has a longitudinal axis generally parallel with the longitudinal axis of a main portion of a cannula. This screw is in communication with a spring which is in an initial position comprising a generally unbiased state. In one embodiment, a portion of the screw (e.g. a threaded body portion) is disposed within the internal diameter of a coil spring and a head portion of the screw is disposed external to the spring. In one embodiment, a peripheral portion of a head of the screw contacts a proximal end of the spring in a manner that allows for force transmitting communication between the spring and the screw. For example, a peripheral portion of the head of the screw may be in contact with a proximal end of a coil spring wherein the contact between the peripheral portion of the screw and the end of the spring does not inhibit rotational movement of the screw, yet provides for the application of a force in a direction generally parallel with the longitudinal axis of the spring and the screw. In one embodiment, a distal portion of the spring is fixedly attached to a portion of the cannula. For example, the cannula or channel in which the spring is disposed may have an annular member which a distal end of the spring is fixedly attached to, thus impeding rotation and linear displacement of the spring at at least one end of the spring.

In an alternative embodiment, the spring is disposed within a cannula (e.g. within a wall-thickness of the cannula) or a channel attached to a cannula and is not anchored or otherwise secured. In this embodiment, the spring is contained between a distal end of the channel and at least a portion of the screw due to the diameter of the spring.

In a preferred embodiment, the present invention comprises a coil spring. One of skill in the art will recognize that a variety of biasing members (whether linear or non-linear) and/or spring constants may be employed. The spring should preferably comprise a sufficient spring constant or restoring force to return a screw to an original position within the channel or cannula when the screw is not secured to a patient and/or external force is not applied to the screw. The spring constant should not be so great as to complicate anchoring of the screw into a patient and similarly should not comprise enough force so as to be capable of shearing or dislodging an anchored screw.

In one embodiment, cannulas of the present invention comprise a plurality of channels and/or spring loaded screws as described herein. Where two or more anchor screws are desired, the channels and anchor screws may be radially distributed around a periphery of the cannula.

In one embodiment, the channel in which the spring/screw combination of the present invention is housed is comprised at least partially of a generally transparent material to allow for visualization of the spring and screw. In one embodiment, a visible portion of the channel is graduated or otherwise marked with indicia capable of displaying information related to the displacement and/or location of the spring and/or screw.

The present invention further comprises various angled tools, distractors and/or impactors which assist in procedures involving a non-perpendicular disk space. For example, it is known that based upon the natural curvature of the spine and the manner in which a patient is situated during surgical procedures, certain vertebrae or other elements to be operated on will be offset from a direction normal to the operating surface. Frequently, in lateral based spinal procedures, a patient will be bent laterally to open space between the ribs and iliac crest. While this method of operation facilities the insertion of dilating cannula and other devices, it typically requires procedures to be performed in a non-perpendicular manner.

In one embodiment of the present invention, angled tools are provided which comprise replaceable and/or disposable working ends. Angled scraping tools are contemplated which comprise detachable ends which may be replaced and/or discarded when edges or ends of the device become dull, soiled, damaged, or are otherwise undesirable for use in further procedures.

Still other embodiments of the present disclosure provide for a distraction tool and/or an impactor or holder having an exterior shaft and a distally-located rotating hinge that is configured to allow the head portion of the distraction tool and/or the distal end portion of the impactor or holder to rotate in at least one plane and thereby be offset from the handle of the distraction tool and/or an impactor or holder, and from the exterior shaft, by any one of any number of angles. The rotating hinge thus allows the head portion of the distraction tool and/or the distal end portion of the impactor or holder to be offset from the handle and from the exterior shaft by an angle so that they may be utilized as described herein without the need for the handle of the distraction tool and/or an impactor or holder or for the exterior shaft to be aligned with the surgical site at a right angle. In some embodiments, the angle of the head portion of the distraction tool and/or the distal end portion of the impactor or holder is determined by securing means located at the distal terminus of the external shaft, which are configured to lock the rotating hinge in place, and thereby set at a desired angle of use for the head portion of the distraction tool and/or the distal end portion of the impactor or holder. In further embodiments, the distally-located rotating hinge is configured to allow for rotation of the head portion of the distraction wedge and/or the distal end portion of the impactor or holder in three dimensions around a single fixed point, in a manner substantially similar to a wave platform shaker.

In one embodiment, the distractor and/or impactor tool shaft comprises a syringe-type or plunger handle which allows for the release of pins or elements disposed within a main body portion of the tools and allows for axial rotation of a lower portion of the tool. A proximal portion of the tool may be further equipped with a flange portion or similar lateral protrusions to facilitate communication with a user's hand and/or fingers. Various features and devices disclosed in U.S. Patent Application Publication No. 2010/0076445 to Pagano, which is hereby incorporated by reference in its entirety, may be utilized in the present invention.

In one embodiment, a rotating/locking hinge of the distractor and/or impactor tool is provided which allows for rotation about at least one axis between an angle of −90 and 90 degrees with respect to a line corresponding to the longitudinal axis of the main body portion of the device. In a more preferred embodiment, the rotating head of the present invention is capable of rotating between −30 and 30 degrees with respect to a line corresponding to the longitudinal axis of the main body portion. Thus, as will be recognized, the combination of the ability to axially rotate the shaft or main body portion of the distractor and/or impactor tool in combination with the ability to pivot or alter the angle of the distal end of the device creates the functionality of a universal joint and allows the working end of the device to rotate to any number of non-discrete desired positions.

In one embodiment, a distal end of the distractor/impactor tool is activated by a thumb screw or similar device located at a proximal end of the device which interacts with the rotating head disposed on a distal portion of the device by a longitudinally extending member such as a rod or screw. In one embodiment, activation of the thumb screw effects rotation of the rotating head in part due to gearing (e.g. offset gears, worm gears, etc.) disposed at a location within the device and between the thumb screw and the rotating head.

In another embodiment, the present invention comprises flexible wires which may be activated at a proximal end of the device and used to control the orientation of a distal portion of the device. U.S. Patent Application Publication No. 2009/0192350 to Mejia, which is hereby incorporated by reference in its entirety, discloses a scope comprising means such as articulating wires for controlling an end of a device.

In one embodiment, one or more biasing members are employed which assist in maintaining engagement between pins and a lower potion or head of the device.

In another embodiment, the present invention comprises a threaded member at the distal end of the distractor/impactor device which is a capable of accommodating any number of additional devices, including, but not limited to distractor devices and final disc or disc replacement implants.

In alternative embodiments, a distal end of the present invention comprises various selectively disengagable attachment means, including, but not limited to clamps, magnets, forceps, and other devices capable of engaging and/or selectively releasing various tools and/or implants.

In one aspect of the present disclosure, a tool is provided for delivering or placing an intervertebral cage, graft, distraction wedge or other device into an implant or surgical site. The tool has a generally syringe-shaped handle and body, with, on the handle, a means for communicating with the distal end of the body. In one embodiment, the means for communication comprises a component manipulated by a surgeon's thumb during an operation. Such means may comprise a track ball, a rotating member, a lever, an electronically controlled signal, a button, or another structural feature that provides desired movement at the opposite end of the tool. In one such embodiment, a thumbscrew or thumbwheel is at the proximal end of the handle, and a hollow, cylindrical body is attached to the handle whereby the thumbscrew can rotatably communicate with the distal end of the body via a cable, shaft or other rotating means. The tool has a hinge on the distal end of the body that rotates in at least one axis that is substantially perpendicular to the axis of the cylindrical body, and that interconnects the body to the head of the tool. The hinge on the distal end provides articulation of the head with respect to the body of the tool. It should be understood that the hinge may be any rotating member that allows rotation in at least one plane between two parts.

The cylindrical body has at least one fastener, at a point between the proximal handle and the distal rotating member, which is capable of securing a tab, tether or other apparatus to the body of the tool. The fastener may be, for example, a clamp, a clip, a cam-lock or other similar device. In yet another aspect of the present disclosure, this fastener may be affixed, or it may be drawn towards the proximal handle, or it may be wound in order to draw in the tab, tether or other apparatus. Devices that may draw in the tab, tether or other apparatus are well known in the art. Incorporated by reference herein in their entireties are the following U.S. patents and publications generally directed to reels and cam-locking devices that may be used to draw in the tab, tether or other apparatus of the present disclosure: U.S. Pat. No. 6,149,096 to Hartley, U.S. Pat. No. 4,039,156 to Abraham, U.S. Pat. No. 3,741,496 to Beller, U.S. Pat. No. 3,836,092 to Hull, U.S. Pat. No. 5,058,823 to Emura, U.S. Pat. No. 5,333,812 to Sato and U.S. Pat. No. 3,697,011 to Christensen.

Although well suited for use in human patients, and although much of the discussion of the present disclosure is directed toward use in humans, advantages offered by the present disclosure may be realized in the veterinary and scientific fields for the benefit and study of all types of animals and biological systems. Additionally, although embodiments of the present disclosure are particularly well-suited for procedures conducted in the spinal column between two target vertebrae, and although much of the discussion of the present disclosure is directed toward use in spinal applications, advantages offered by embodiments of the present disclosure may also be realized by implantation at other locations within a patient where the fusion of two or more bony structures may be desired. As one of skill in the art will appreciate, the present disclosure has applications in the general field of skeletal repair and treatment, with particular application to the treatment of spinal injuries and diseases. It should be appreciated, however that the principles of the present disclosure can also find application in other areas, specifically where there is a desire to constrain added fluid material to particular regions. For example, the present disclosure finds application in methods where the objective is to confine added material to predetermined areas of interest and to prohibit the undesired translocation of such material until an operation is complete and/or until a predetermined later time.

In various embodiments, one or more cannulas of the present invention comprise features for selectively expanding a distal end and/or expanding a work or operative site. U.S. Pat. No. 7,682,370 to Pagliuca et al. relates to an expanding cannula or tubular structure for receiving surgical instruments, and is hereby incorporated by reference in its entirety. Specifically, the present invention contemplates features for introducing a cannula or device within a patient and/or to a workspace in a minimally invasive fashion and further expanding particular portions of the cannula or device to maximize work space or visualization.

In one embodiment, the present invention comprises a cannula for minimally invasive surgery that is expandable at specific predetermined regions. U.S. Pat. No. 6,613,038 to Bonutti et al. relates to a method of using expandable cannula and is hereby incorporated by reference in its entirety. Bonutti, however, relates to a cannula that expands along its entire length. While such features may be advantageous for use in vascular stents, for example, the present invention pertains to the notably distinct field of minimally invasive surgery where it is undesirable or unacceptable to expand an entire length of a cannula. Accordingly, in various embodiments, the present invention contemplates a device having a sufficiently small initial diameter such that it may be inserted in a minimally invasive percutaneous fashion and adapted to expand in specific regions based on user discretion/input. For example, in one embodiment, a cannula is provided having segments which may be hinged or pleated and expand outwardly at a user's discretion in order to create more space at or near a surgical target site or at a user-proximal location in order to create greater freedom of movement for tools and enhance visualization.

U.S. Pat. No. 7,693,562 to Marino et al., which is hereby incorporated by reference in its entirety, discloses an expandable tip cannula system having an expandable distal tip.

In one embodiment, a system for performing spine surgery is provided, the system comprising a surgical access instrument deliverable along a lateral, trans-psoas path to a targeted spinal site to create an operative corridor. The surgical access instrument has a proximal portion, a distal portion, and an elongate length with a longitudinal axis. In one embodiment, the distal portion has an asymmetric cross sectional shape with a convex portion positioned and adapted to extend or enlarge an operative corridor away from known areas of high nerve concentration.

In various embodiments, a distal portion has at least one nerve stimulation electrode located for sensing the proximity of nerves. The system may further comprise feedback features for alerting a user of the proximity (e.g. unacceptable proximity) of nerves and/or provide a signal that impingement upon nerves is likely. The convex portion is selectively and radially expandable from a first initial position, such that a workspace may be enlarged only in directions or areas extending away from various nerves.

In various embodiments, the system comprises one or more piezoelectric materials to which current may be applied in order to change the shape or enlarge various specific portions of a surgical access instrument (e.g. cannula, working cannula, etc.).

In various embodiments, both a distal and a proximal portion of the device are selectively expandable. Accordingly, in at least one embodiment, a surgical instrument is provided that can be enlarged both at a location proximal to a work site and at a location proximal to a user, thereby creating an “hour glass” type device that allows for freedom of movement of surgical tools at proximal and distal locations while passing through a narrow (i.e. minimally invasive) portion of the device which is generally disposed within skin and/or tissue. Selective enlargement of various portions of a device may be accomplished by, for example, segmented hinged or pleated portions which may are disposed in a closed or “narrow” arrangement in a first or insertable position. The segmented hinged or pleated portions may thereafter be selectively expanded or opened in order to provide additional work space while maintaining minimally invasive aspects of the present invention. Various portions of the device, particularly those adapted to move or expand, may include nerve sensing features as discussed herein.

As will be recognized by one of ordinary skill in the art, it is desirable in the field of minimally invasive surgery to provide secure and/or enclosed access to a target site. In contrast to the aforementioned Marino et al., which contemplates hinged portions adapted for the expansion of the tip of a cannula, the present invention further contemplates an expandable tip which additionally provides secure or enclosed expansion of various portions of a cannula or dilator. Various embodiments of the present invention contemplate expanding an operative corridor without creating gaps, perforations, or openings through which fluid and tissue may enter or encroach upon the interior of a tool and/or the operative corridor. To this end, pleats, foldable portions, and membranes may be employed to allow for secured expansion of a portion (e.g. distal portion) of the device.

In one embodiment, a convex portion of an asymmetric cannula comprises hinged sections, adapted to be selectively expanded or enlarged and thereby enlarge a surgical workspace wherein the convex portion further comprises an elastic member or material adapted for expanding in connection with the expansion of the hinged sections. Thus, when hinged portions are activated or expanded, gaps otherwise created by such expansion are accounted for by the elastic member or material and prevent unwanted encroachment of various materials, tissues, and fluids.

These and other advantages will be apparent from the disclosure of the invention(s) contained herein. The above-described embodiments, objectives, and configurations are neither complete nor exhaustive. As will be appreciated, other embodiments of the invention are possible using, alone or in combination, one or more of the features set forth above or described in detail below. Further, this Summary is neither intended nor should it be construed as being representative of the full extent and scope of the present invention. The present invention is set forth in various levels of detail in this Summary, as well as in the attached drawings and the detailed description below, and no limitation as to the scope of the present invention is intended to either the inclusion or non-inclusion of elements, components, etc. in this Summary. Additional aspects of the present invention will become more readily apparent from the detailed description, particularly when taken together with the drawings, and the exemplary claim provided herein.

### BRIEF DESCRIPTION OF THE FIGURES

FIG. 1 is a cross-sectional illustration of a patient illustrating features and operation of prior art systems;

FIG. 2 is a front elevation view depicting various features of the present invention;

FIG. 3 is a cross-sectional view of various features of the present invention;

FIG. 4 is a cross-sectional illustration of a patient illustrating features and operation of the present invention;

FIG. 5 is a cross-sectional illustration of a patient illustrating features and operation of the present invention;

FIG. 6 is a cross-sectional illustration of a patient illustrating features and operation of the present invention;

FIG. 7 is a front elevation and a cross-sectional view of another feature of the present invention;

FIG. 8 depicts various tools that may be employed with embodiments of the present invention to assist with disk space distraction and preparation;

FIG. 9 is a cross-sectional elevation view of a patient wherein features of the present invention are utilized; and

FIG. 10 is a front elevation view of another aspect of the lateral based retractor system;

FIG. 11 is a front view of a working cannula according to one embodiment of the present invention.

FIG. 12 is a front view of a working cannula according to one embodiment of the present invention.

FIG. 13 is a top view of a cannula according to one embodiment of the present invention.

FIG. 14 is a top view of a cannula according to one embodiment of the present invention.

FIG. 15 is a perspective view of a fiber optic cannula according to one embodiment of the present invention.

FIG. 16 is a top view of a fiber optic cannula according to one embodiment of the present invention.

FIG. 17 is a perspective view of a fiber optic cannula and working cannula according to one embodiment of the present invention.

The drawings are not necessarily to scale.

### DETAILED DESCRIPTION OF THE FIGURES

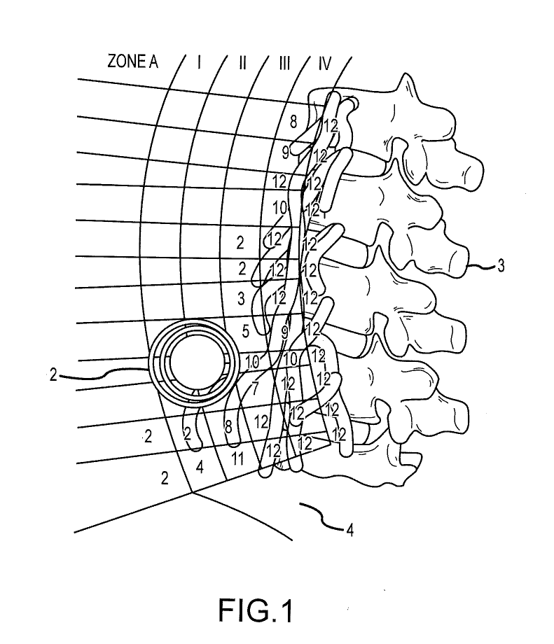

FIG. 1 is a cross-sectional illustration of a patient illustrating features and operation of prior art systems. FIG. 1 includes a patient's spinal column 3, various nerves (e.g. femoral, psoas) 4, and a lateral spinal surgery system 2 according to known devices and represented by concentric circles. This device and those similar may be operated by progressively inserting cannulas 2, beginning with the smallest and sequentially increasing the cavity by expanding muscle and tissue (e.g. the psoas) via the insertion of larger cannulas. As illustrated in FIG. 1, concentrically expanding from a desired center point of a surgical area poses the risk of impinging upon various nerves 4. With reference to FIG. 1, expansion from a center in a radially uniform manner poses increased risk for impinging upon nerves. Various complications, including nerve damage and post-operative leg pain, are associated with this impingement. The present invention provides novel devices and methods of use which contemplate cannulas and other surgical tools consisting of desirable cross-sectional shapes that further reduce the risk of impinging upon nerves and tissue when inserted into and maintained within a surgical workspace.

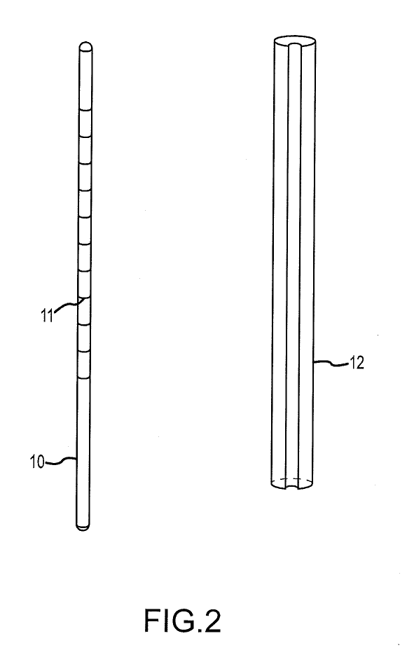

FIG. 2 is a front elevation view depicting various features of the present invention. A pilot cannula 10 is shown which, in one embodiment, includes depth indication markers 11 in 10 millimeter increments. It will be recognized that the present invention is not confined to a pilot cannula 10 with depth indication at any specific increments or with any depth indication provided. However, as the present invention is contemplated for use in minimally invasive procedures, it is often desirable to provide various user feedback features to compensate for the generally reduced visibility of the surgical area when compared to open surgeries. It will be expressly understood that a pilot cannula, pilot member, or guide member 10 of the present invention is limited to any particular device. Rather, the invention contemplates a member 10 that is of sufficiently small dimensions to fulfill its intended purpose and is therefore not limited to devices that are solid, hollow, etc.

In one embodiment, a guide pin (now shown) may be inserted percutaneously. This guide pin may be of sufficiently small dimensions so as to minimize the risk of injury to the patient, particularly where it may need to be extracted and reinserted before finding its optimal location. A pilot cannula may further be provided that may be inserted around the guide pin. In one embodiment, the pilot cannula 10 or guide pin include(s) sensing means such as wires and a conductive tip that are operable to sense the proximity or lack thereof of nerves. In this manner, a desired surgical area may be evaluated before expanding the area to its ultimate desired size. Accordingly, injury to the patient may further be reduced.

After a surgical area has been selected and evaluated, devices of the present invention may be employed, including, but not limited to, sequentially larger dilator cannulas. These dilator cannulas may gradually increase the size of a desired surgical area with reduced risk of impinging upon nerves. As will be described further, the dilator cannulas may comprise a cross-sectional shape that provides for non-concentric radially non-uniform expansion of a surgical area. As those working in the art will recognize, conductive features for sensing nerve proximity may be incorporated on various aspects of the present invention and are not limited to the pilot cannula 10.

FIG. 2 also depicts a dilator cannula 12 according to one embodiment of the present invention. The accompanying cross-section of the dilator cannula 12 reveals an asymmetry which is a novel feature of the present invention. These cannulas, which may be inserted in progressively larger sizes (as will be described later), are constructed or formed in this manner so as to avoid or mitigate complications presented with the prior art (as described above). Specifically, the insertion of these cannulas with asymmetric cross-sections allows a user to expand the surgical area without expanding into a specific region(s) where the device may impinge upon nerves and other tissue.

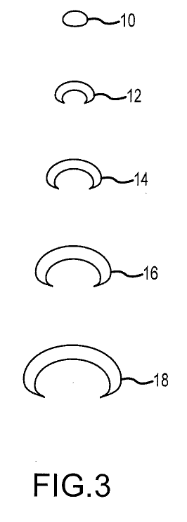

FIG. 3 depicts cross-sectional views of various cannulas 10, 12, 14, 16, 18 and is further illustrative of how these aspects of the present invention interoperate. Although FIG. 3 depicts a single pilot cannula 10 and four dilator cannulas 12, 14, 16, 18, one of ordinary skill in the art will recognize that the present invention is not limited to any specific number or shape of cannula. It will further be recognized that cannulas of different cross-sections would also serve the spirit of the present invention, so long as that when cannulas of increasing size are inserted, these larger cannulas do not expand the surgical area uniformly in all radial directions. For example, cannulas of triangular, elliptical, ovoid, egg-shaped or other similar cross-sectional shapes may also achieve objects of the present invention. In various embodiments, features of the present invention comprise conical, bullet-shaped, or tapered distal ends so as to facilitate their insertion and expand tissue rather than cutting or tearing and thereby reducing injury to a patient.

Additionally or in place of a guide pin, a pilot cannula of slightly larger diameter may be inserted. This pilot cannula may either be placed around or proximal to the guide pin. The pilot cannula may further include means, such as conductive wires and sensors, to detect the proximity of nerves and nerve structures. Guide pins and/or pilot cannula(s) may further be used to direct the location of various dilators which may gradually expand the desired surgical area in a non-concentric or radially non-uniform fashion so as to allow adequate room for surgical tools and procedures, with reduced risk of impinging upon nerves. Specifically, dilators of the present invention are shaped in an asymmetric fashion so as to expand the surgical area in a defined radial direction(s) and to avoid impingement of the areas with higher concentrations of nerve structures.

Various devices of the present invention include anchoring means, such as screws and/or tangs and stakes to secure the devices once located in a desired position. These anchoring means further operate to reduce the risk of injury to the patient as repositioning or re-insertion of these and similar devices further increase the risk of damaging surrounding nerves and tissue. Once a sufficient degree of dilation of a surgical area is achieved, smaller cannula disposed within the larger cannula may be removed to allow for visibility and usable space within devices of larger diameters. Devices of larger diameters, such as dilators of an appropriate size, retractors, and working cannulas may be employed to stabilize the surgical area. These devices, including an outer working cannula, may be secured by previously discussed anchoring means or by external mounting devices. Outer working cannula, as will be apparent from the present disclosure, may include various additional features such as fiber-optic lighting elements, imaging equipment, luminescent materials, bioluminescent materials, etc.

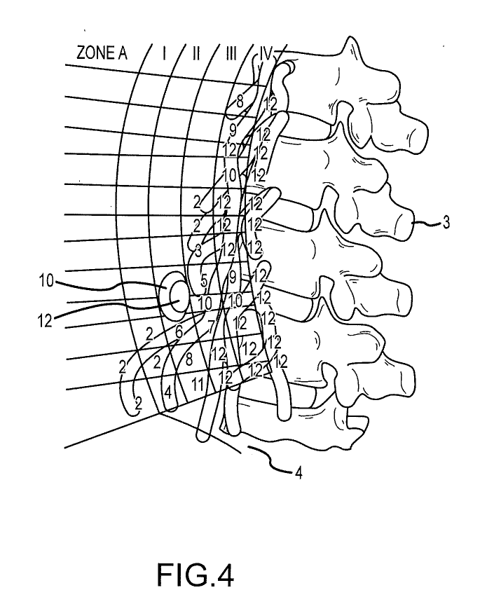

FIG. 4 is a cross-sectional illustration of a patient illustrating features and operation of the present invention. FIG. 4 illustrates features of the previously discussed embodiments with reference to a patient's spine 3 and nerve structure 4. As one of ordinary skill in the art will recognize, the present invention allows for expansion and enlargement of the surgical area in a non-uniform manner. Accordingly, as shown in FIG. 4, enlargement of a surgical cavity may be achieved without impinging upon nerves or specific regions of a patient.

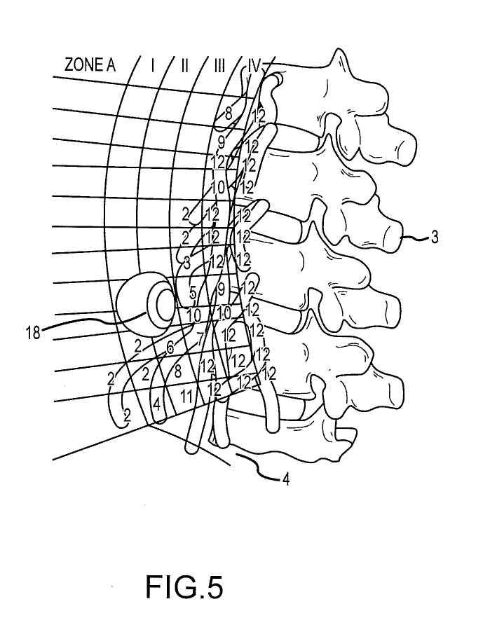

FIG. 5 is another cross-sectional illustration of a patient illustrating features and operation of the present invention. FIG. 5 depicts multiple asymmetric cannulas in relation to a patient's spine 3 and nerve structure 4. Once the working cannula 18 (i.e. the cannula of largest desired diameter) is secured, remaining dilator cannulas are removed. In this manner, the psoas and other tissues are parsed so as to allow for access for spinal surgery and a working aperture is provided. It will be recognized that the working cannula need not be of any specific size or order to achieve the previously described sequence. Various factors, including patient size, procedure(s) to be performed, and/or tools to be used may determine the appropriate size of the working cannula.

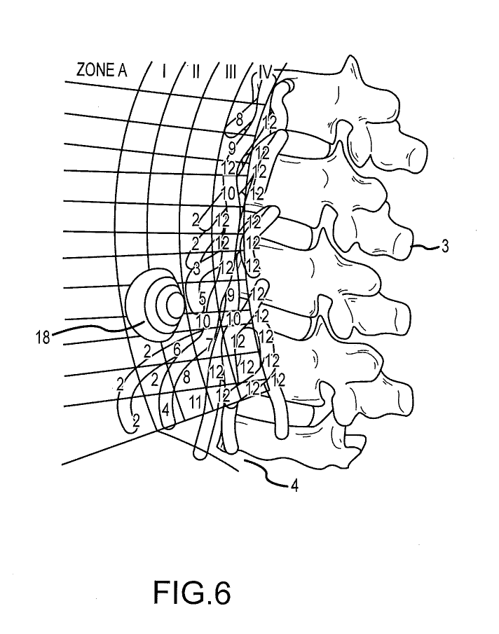

FIG. 6 is a cross-sectional illustration of a patient wherein the non-working dilator cannulas have been removed from the cavity. FIG. 6 further illustrates a formed surgical cavity of sufficient size with minimal impingement upon the psoas and other nerves. Specifically, as consecutively larger devices are inserted, expansion is achieved in a direction away from areas III and IV as shown in FIG. 6. Expanding the work area in this manner provides for reduced risk of impinging upon nerves by avoiding areas of the greatest concentration of nerves.

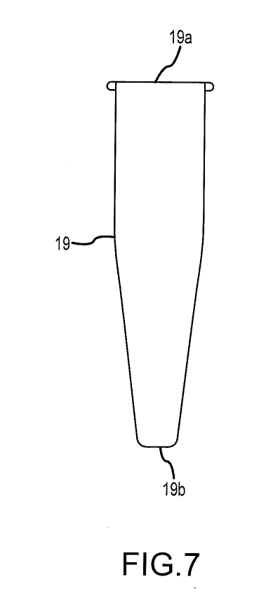

FIG. 7 depicts a psoas retractor deploying tool 19 according to one embodiment of the present invention. An asymmetrical cross-section according to one embodiment is also shown. The elevation view of the retractor deploying tool 19 illustrates a tool with graduated thickness (i.e. a device with at least one partially conical section). While the present invention is not limited to any specific dimensions, it is desirable to provide a tool with a base diameter 19b or width of approximately 1.5 cm and a proximal or user end 19a with a diameter or width of approximately 3.0 cm. Although dimensions may be modified without violating the spirit of the present invention, those working in the art will recognize that it is desirable to provide a distal end with an aperture of sufficient diameter to perform various surgical procedures including, but not limited to, lateral interbody fusion and disk repair/replacement procedures.

Various features of the present invention contemplate the use of a set screw(s), staple, or other anchoring means to secure one or more tools once successful insertion has occurred. As those working in the art will understand, it is desirable to stabilize various tools and devices which define a surgical working area. Various prior art devices do not teach an anchoring means and therefore suffer from inadequate refractor fixation. Inadequate retractor fixation which results in movement of the lateral based spinal surgery devices and translation from their initial position often necessitates removal and reinsertion of devices. Repeated removal and reinsertion is believed to cause, or at least increase the risk of, nerve and tissue damage, often resulting in neuropraxia and leg pain. Accordingly, the present invention contemplates means including external fixation and/or stabilization of cannula(s) as well as anchoring screws to inhibit movement of devices once placed in their desired locations.

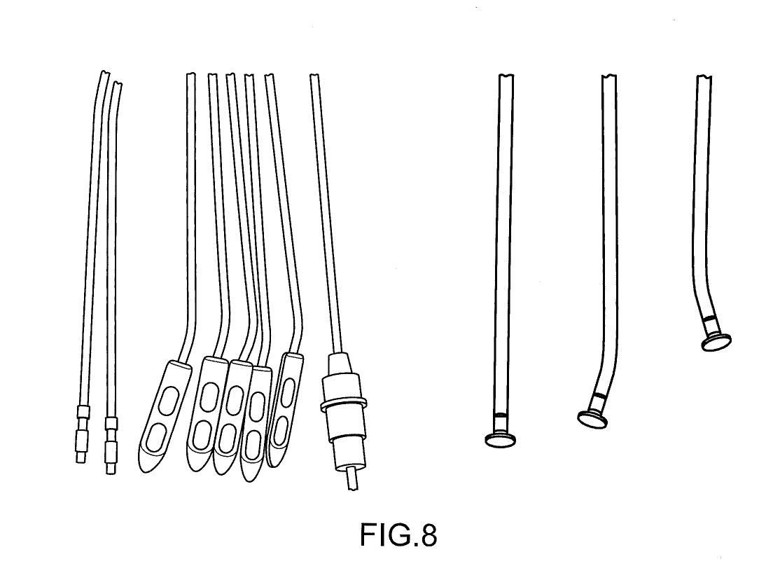

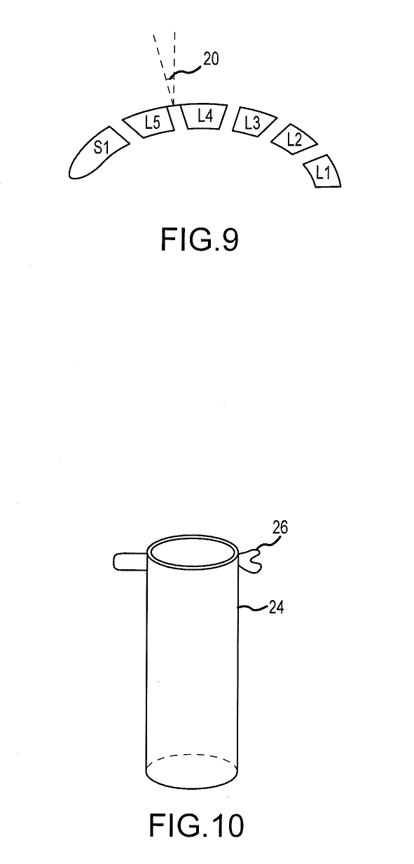

FIG. 8 depicts various tools that may be employed with embodiments of the present invention to assist with disk space distraction and preparation. These devices are representative of some of the surgical tools that may be utilized in conjunction with previously described embodiments of the invention. Various angled instruments are shown which assist in procedures involving a non-perpendicular disk space. For example, it is known that based upon the natural curvature of the spine and the manner in which a patient is situated during surgical procedures, certain vertebrae or other elements to be operated on will be offset from a direction normal to the operating surface. Frequently, in lateral based spinal procedures, a patient will be bent laterally to open space between the ribs and iliac crest. While this method of operation facilities the insertion of dilating cannula and other devices, it typically requires procedures to be performed in a non-perpendicular manner as shown in FIG. 9.

FIG. 9 depicts different angles of approach 20, 22 for various different minimally invasive procedures. As will be recognized, certain procedures are conducive to surgical procedures occurring substantially parallel to spinal discs. Alternatively, lateral entrance in minimally invasive procedures is often employed where the work area is considered a non-perpendicular surgical location 22. Features of the present invention contemplate providing angled distractors and/or impactors which may be utilized in conjunction with various previously described cannulas and further provide for increased accessibility to non-perpendicular surgical locations (e.g. between L4 and L5). One of the benefits of this aspect of the present invention is the reduced risk of damaging spinal endplates.

FIG. 10 is a front elevation view of a fiber optic cannula 24 for use with various embodiments of the present invention. Prior art cannula and surgical techniques sometimes lack direct lighting or provide video imaging of the surgical workspace. While the present invention is not limited to employing these devices in any particular location, one embodiment contemplates providing an additional cannula that is operable with or as the working cannula(s) 24. This device, according to one embodiment, includes fastening means 26 to attach and secure the device to either a table attachment or to secure devices already being utilized in a procedure. Furthermore, the device 24 is designed to be able to communicate with various fiber optic devices including light sources and/or imaging equipment. It is contemplated that this device may be comprised of a light conductive plastic or similar device that is disposable, so as to transmit light from one portion of the device to a surgical area or another portion of the device.

The present invention further contemplates a method of use for various embodiments described herein. In one embodiment, minimally invasive surgical procedures may be performed utilizing features of the present invention whereby consecutively larger cannulas are inserted into a patient to both create and maintain a working surgical area. Specifically, a procedure may be initiated by a guide pin or needle. A pilot cannula or guide device comprising a conductive wire for sensing the proximity of nerves may also be inserted into a work area in conjunction with or oriented around the guide pin. The pilot cannula or guide device may be appropriately positioned so as to provide a reference point around which various cannulas as described herein may be inserted to gradually increase the size of the surgical area. One of skill in the art will recognize that these cannulas need not be of a specific number or size. Rather, the present invention contemplates the use of as few as one cannula and as many as may be desired based upon various considerations including but not limited to the desired size of the surgical area, the size of the patient, and the procedure to be performed. It will also be recognized that the present invention is not limited to any specific sequence with respect to the above described devices. For example, the present invention contemplates a method wherein a pilot cannula may be the first device employed, or wherein a pilot cannula is inserted subsequent to a guide pin.

In one embodiment, this method for minimally invasive surgical procedures further includes anchoring means located on one or more of the cannula to secure disclosed devices once they are located at a desired position. These anchoring means may be comprised of one or more screws, stakes, pins, and other similar devices. In another embodiment, a method of use further comprises light sources provided in association with devices of the present invention. For example, a fiber optic light source may be provided at a distal end of one or more of the inserted cannulas or devices of the present invention may be comprised of a light conductive material that is conducive to transporting light along cannulas and other surgical devices to a surgical area.

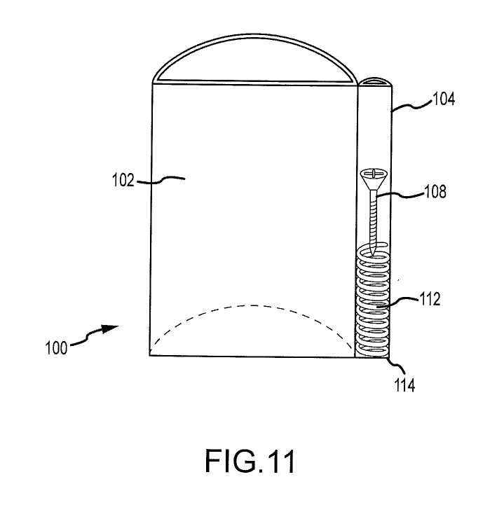

FIG. 11 is a front view of a feature of one embodiment of the present invention. As shown in FIG. 11, an asymmetric working cannula 100 is provided which comprises a generally cylindrical housing 104 that has a longitudinal axis generally parallel to the longitudinal axis of the main body portion 102 of the working cannula 100. The cylinder housing 104, in one embodiment, comprises a fastener. In one embodiment, the fastener, such as a bone screw 108 is provided which has a threaded portion with an outer diameter less than an inner diameter of a corresponding coil spring 112 or similar biasing element. The bone screw 108 has at least one dimension (e.g. the outer diameter of the head of the screw) which is greater than the diameter of the coil spring and thus prevents the bone screw 108 from translating or passing through the coil spring 112. Thus, the bone screw 108 may be at least partially disposed within the coil spring 112, yet the spring and a portion of the bone screw 108 are capable of contacting an imparting force upon one another. The cylinder housing 104 comprises a proximal end which allows for user access to a bone screw 108 and/or the coil spring 112. This access, for example, may be accomplished by the insertion of a screw driver (not shown) into the cylinder housing 104 which is further capable of contacting a bone screw 108. Thus, when a user desires to affix the working cannula to a patient in a given position, a screw driver or similar device may be inserted into the cylinder housing 104, may impart force upon the bone screw 108, translating the bone screw 108 to a distal position within the cylinder housing 104, and placing the bone screw 108 in contact with the patient at a desired location. It will be recognized that when the bone screw 108 is translated toward the patient, the coil spring 112 will reside in a compressed state and may be fixed in a compressed state by actuating the bone screw 108 and anchoring the bone screw 108 into the patient. The working cannula 100 may be removed from a patient and/or relocated upon disengaging the bone screw 108 from the patient through the use of a screw driver or appropriate tool.

It is to be expressly recognized that although the cylindrical housing 104 is depicted as a generally enclosed pathway, the present invention is not limited to this arrangement. Rather, the present invention contemplates various channels or guiding features capable of directing, holding, or guiding a screw or fastener as (e.g. a bone screw). Thus, the cylindrical housing 104 may be performed, partially open, slotted, etc. In a preferred embodiment, however, the cylindrical housing is substantially enclosed so as to isolate various features from tissue and fluids.

One of skill in the art will recognize that when the screw is disengaged from the patient, the coil spring 112, which was previously positioned in a compressed state, will translate the bone screw 108 toward an original position within the cylinder housing 104, thereby reducing or eliminating the need to manually extract a bone screw from a surgical workspace by hand or through the use of additional tooling. Furthermore, risk of loss of the screw and/or injury to the patient is reduced through the implementation of a contained spring-loaded screw. Thus, manipulation of both a screw driver or similar tool and a bone screw within a surgical work space is greatly simplified.

Although FIG. 11 depicts a working cannula 100 with a single cylinder housing and associated bone screw 108 and coil spring 112, any number of cylinder housings as described herein may be disposed on the present invention. For example, cylinder housings 104 may be dispersed radially around at least a portion of the circumference or outer portions of a working cannula. Furthermore, cylinder housings 104 need not be of equivalent longitudinal length as the working cannula itself. Furthermore, cylinder housings 104 of the present invention may be disposed within an interior wall of a working cannula, within an inner-diameter of a working cannula, external to the outer wall of the working cannula, or at a variety of locations therebetween.

In one embodiment, the coil spring 112 is anchored or affixed at one or more portions of the cylinder housing 104. For example, the coil spring 112 may be fixedly attached at a distal portion 114 of the cylinder housing 104. In this embodiment, at least one end of the coil spring 112 is prevented from rotating when bone screw 108 is turned or otherwise activated. The head of the bone screw 108 may engage a proximal portion of the coil spring in such a manner so that rotational movement of the head of the bone screw 108 is not inhibited and/or does not create substantial angular displacement of the coil spring 112. In one embodiment, the coil spring 112 comprises a cap portion at a proximal end which provides for a reduced coefficient of friction between a portion of the bone screw 108 and the coil spring 112. In addition to cylindrical housings 104 for bone screw/spring devices, the present invention contemplates the use of additional similar channels for the implementation of nerve sensing devices. For example, various nerve sensing devices and features known to those of ordinary skill in the art, including but not limited to those described in U.S. Patent Application Publication No. 2010/0076335 to Gharib et al., which is hereby incorporated by reference in its entirety, may be utilized with the present invention to determine nerve proximity and nerve direction, as well as assess the pathology of a nerve or nerve root before, during, or after a surgical procedure. In one embodiment, pathways or guide-channels are provided along a length of the device 100 within which electrical stimulation leads or similar nerve-sensing devices may be inserted or may reside.

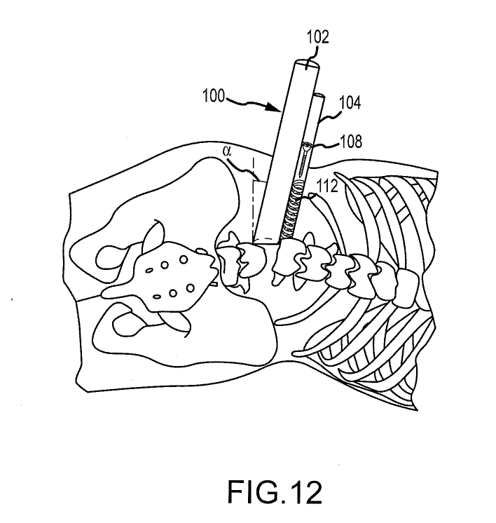

FIG. 12 is a front view of a working cannula according to one embodiment of the present invention. As shown, a distal end opening (i.e. working end) of the cannula 100 is disposed at an angle a from a line normal to a horizontal workspace. In one embodiment, a comprises an angle between 8 and 15 degrees. In a more preferred embodiment, a comprises an angle between approximately 11 and 13 degrees. In a more preferred embodiment, a comprises an angle of approximately 12 degrees. Although the present invention is not limited to any specific angle or angles, it will be recognized that there generally exists an optimal angle of approach when performing various lateral based procedures.

FIG. 12 further displays a working cannula with at least two cylindrical housings 102, 104 and screw/spring 108/112 combinations disposed therein. As previously described, the present invention contemplates cannulas with only a single cylindrical housing 104 as well as cannulas with a plurality of cylindrical housings 104. Cylindrical housings 104 may be disposed generally parallel with a main portion 102 of the device, or may be oriented at a different angle than the main portion 102 of the device.

In one embodiment, the working cannula 100 is of a diameter such that, when properly oriented, the inner space or volume 102 of the cannula 100 provides access to a disc space yet is not sufficiently large to allow for the unwanted entrance of various soft tissue, fluids, etc., from entering and obscuring an operative site. In one embodiment, the internal diameter or width of the working cannula 100 is only marginally larger than the longitudinal length of a disc space.

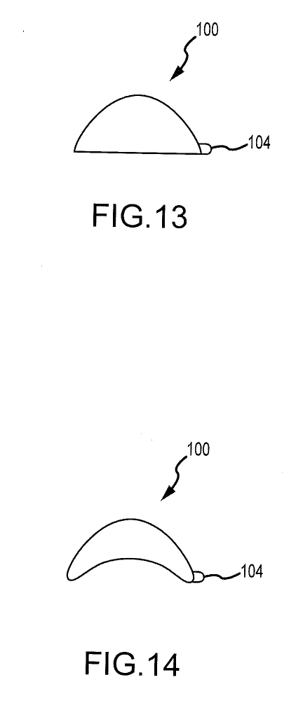

Referring now to FIGS. 13-14, top plan views of various embodiments of working cannulas are provided. As shown, working cannulas 100 are provided with at least one cylindrical housing 104 disposed on an exterior portion of the working cannula 100. As will be recognized, and as is described herein, working cannulas are not limited to any particular asymmetric geometry. Thus, D-shaped cross sections, U-shaped cross sections, and a variety of other combinations and alternatives, including crescent-shaped cross-sections, to the cross sectional shapes are contemplated for use with working cannulas of the present invention.

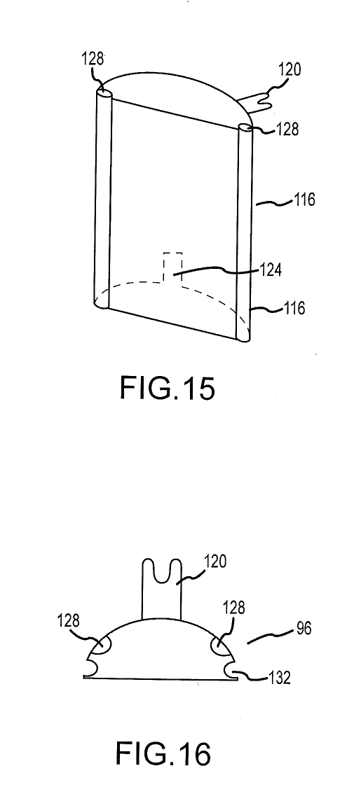

FIG. 15 depicts a fiber optic cannula 116 which is adapted for use with working cannulas as shown and described herein. The fiber optic cannula 116 comprises an attachment portion 120 suitable for securing the fiber optic cannula and connected cannulas to a variety of stationary devices. The fiber optic cannula further comprises fiber optic portals 128 which are capable of accommodating a variety of fiber optic lighting and image sensing devices. In one embodiment, the fiber optic cannula further comprises a compression gap 124 which facilitates the fiber optic cannula 116 in expanding, compressing, and/or locking onto various working cannulas.

FIG. 16 depicts a top plan view of a fiber optic cannula of the present invention, including fiber optic portals 128 and ridges 132 which are adapted to guide a screw driver or similar device into the cylinder housing of an attached or corresponding working cannula. As will be described, fiber optic cannulas of the present invention may be adapted to dock with a working cannula or similar device. Thus, the present invention contemplates features such as ridges or recessions 132 which do not impede or obstruct access to features of the working cannula, such as cylindrical housings and screw/spring assemblies. In one embodiment, guides 132 comprise fully-enclosed cylindrical channels for receiving and guiding a screwdriver or similar tool. Where guides 132 comprise fully-enclosed cylinders, at least a portion of the guides 132 may be comprised of a generally transparent material to display or reveal information regarding the displacement and/or position of objects disposed within. Transparent portions of the guides 132 may further comprise graduation, markings, and/or other indicia.

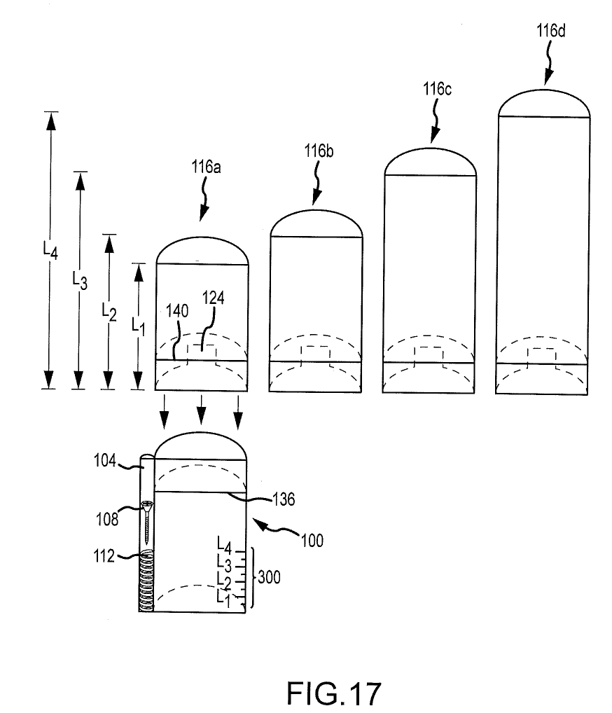

FIG. 17 depicts a working cannula 100 and a plurality of extension cannulas, 116a, 116b, 116c, 116d, which may be fiber optic cannulas. In one embodiment, the working cannula 100 and a fiber optic cannula of the present invention comprise means for engaging one another and/or maintaining communication between the fiber optic cannula 116 and the working cannula 100.

For example, in one embodiment the working cannula 100 comprises a docking ridge 136 which comprises a generally annular protrusion from either a portion or the entirety of the outer circumference of the working cannula 100. The fiber optic cannula 116 may comprise a corresponding ridge or recession 140 at a variety of locations along its longitudinal length, which is adapted to receive the docking ridge 136 of the working cannula 100. In one embodiment, the outer diameter of the docking ridge of the working cannula is greater than both the outer diameter of the main portion of the working cannula and the inner diameter of the fiber optic cannula.

The fiber optic cannula is capable of expanding, for example, due to the inclusion of a compression gap 124. Thus, the fiber optic cannula may be elastically expanded around both the working cannula 100 and the docking ridge 136, and be slid or translated into a position where the docking ridge mates or corresponds with the receiving portion of the fiber optic cannula 116. In an alternative embodiment, docking between a fiber optic cannula 116 and a working cannula 100 is accomplished through one or more non-annular or non-rib like protrusions. For example, one or more protuberances of various sizes and shapes may be provided which interact and dock with corresponding features (e.g. female receiving portions) of a fiber optic cannula 116. One of skill in the art will recognize that receiving (or female) and corresponding (male) docking members may be disposed either on the working cannula 100 and/or the fiber optic cannulas 116a, 116b, 116c, 116d and the present invention is not limited to an embodiment wherein male or female docking portions reside on only one of the contemplated devices. Indeed, male and female portions may reside on either the working, fiber optic, or both cannulas.

In one embodiment the compression gap of the fiber optic cannula 116 relies on the elastic abilities of the fiber optic cannula 116 to expand, accommodate a working cannula 100, and retract to (or approximately to) an initial position. In alternative embodiments, fiber optic cannulas 116 of the present invention comprise a compression gap 124 and/or additional devices useful for and capable of restricting an outer dimension of the fiber optic cannula 116 and thereby securing or attaching the fiber optic cannula 116 to a working cannula 100 or similar device. For example, various known devices may be employed to restrict an outer dimension of the fiber optic cannula, including but not limited to various straps comprising buckles, Velcro, teeth and receiving portions and adjustable strap fasteners.

As further shown in FIG. 17, a plurality of fiber optic cannulas 116a, 116b, 116c, 116d may be provided, each having a different length (L₁, L₂, L₃, L₄). Different lengths are provided in order to provide for a final combined structure (i.e. 100 and 116 combined) with a proximal end disposed at an appropriate distance from a patient and skin edge. In one embodiment, indicia 300 are provided on a working cannula 100 which, when inserted into a surgical work area to the appropriate depth, reveal the corresponding fiber optic cannula which should be utilized.

The present invention further contemplates a method of use of various devices described herein. In one embodiment, lateral based procedures are performed by utilizing progressively larger asymmetric cannulas as described herein as well as working and fiber optic cannulas as described herein. In one embodiment, a method is contemplated where progressively larger cannulas are employed to expand or prepare a surgical workspace. Once the final dilator is applied, the distance from the skin edge to the surgical site is determined by reading a depth line corresponding to the skin edge dilator intersection. This measurement is then used to assemble the appropriate length fiber optic cannula to the working cannula. The working cannula is designed for multi-procedure and has a fixed length of approximately 5 cm. The fiber optic portion of the cannula, which is anticipated to be disposable, comes in 1 cm graduated lengths. The appropriate disposable fiber optic cannula is attached to the working cannula, creating an assembled length equal to the measurement determined by the final dilator. The assembled unit is then slid, placed, or otherwise disposed over the final dilator. Once in position, nerve sensing means and/or a securing screw is stimulated to determine whether or not the assembled unit is disposed over nerve tissue. The screw used to secure the assembly to the patient's vertebra can then be safely deployed and the dilator device removed. This leaves a safe conduit from the skin edge to the disc space. The combined assembly is then secured to a fixed external structure to further secure and stabilize devices of the present invention. Once secured, fiber optic lighting and/or image sensing devices may be activated and procedures may be preformed within the area provided by and defined by aforementioned devices.

While various embodiments the present invention have been described in detail, it is apparent that modifications and alterations of those embodiments will occur to those skilled in the art. However, it is to be expressly understood that such modifications and alterations are within the scope and spirit of the present invention, as set forth in the following claims. Further, the invention(s) described herein are capable of other embodiments and of being practiced or of being carried out in various ways. In addition, it is to be understood that the phraseology and terminology used herein is for the purposes of description and should not be regarded as limiting. The use of “including,” “comprising,” or “adding” and variations thereof herein are meant to encompass the items listed thereafter and equivalents thereof, as well as, additional items.

What is claimed is:

1. A system for accessing a surgical target site, comprising:
a plurality of sequentially sized dilators deliverable along a lateral, trans-psoas path to a targeted spinal site to create an operative corridor;
the plurality of sequentially sized dilators having a first end, a second, and a longitudinal length therebetween, said two or more dilators comprising an asymmetric cross-sectional shape;
a pilot member being configured to introduce said two or more dilators along a lateral, trans-psoas path toward the target site to create an operative corridor to said surgical target site; and
a working cannula that maintains said operative corridor along said lateral, transpsoas path to a targeted spinal site, the working cannula having an inner surface defining a path for receiving surgical instruments.

2. The system for accessing a surgical target site of claim 1, wherein the working cannula comprises a main body portion and a secondary portion;
the secondary portion having a length substantially similar to the length of the main body portion and a longitudinal axis being substantially parallel to the longitudinal axis of the main body portion;
wherein the secondary portion is positioned adjacent to a portion of the outer surface of the main body portion; and
wherein the secondary portion is adapted to receive a fastener.

3. The system for accessing a surgical target site of claim 1, wherein at least one of said plurality of sequentially sized dilators comprise PEEK.

4. The system for accessing a surgical target site of claim 1, wherein the working cannula comprises indicia on an outer surface, said indicia providing information relating to members adapted for extending the length of the working cannula.

5. The system for accessing a surgical target site of claim 1, wherein when said plurality of sequentially sized dilators are delivered along a lateral, trans-psoas path, the operative corridor is enlarged in a manner characterized by radial non-uniformity.

6. The system for accessing a surgical target site of claim 1, wherein said working cannula comprises distal end having an aperture, the aperture being oriented at an angle from the longitudinal axis of the working cannula of approximately between 75 and 82 degrees.

7. The system for accessing a surgical target site of claim 1, wherein said working cannula comprises distal end having an aperture, the aperture being oriented at an angle from the longitudinal axis of the working cannula of approximately between 77 and 79 degrees.

8. The system for accessing a surgical target site of claim 1, wherein said system comprises a plurality of extension cannulas adapted to operably connect to said working cannula in a manner that allows for variable longitudinal extension of said working cannula.

9. The system for accessing a surgical target site of claim 1, wherein said working cannula comprises a selectively expandable distal portion.

10. The system for accessing a surgical target site of claim 1, wherein said pilot member comprises means for sensing the proximity of nerves.

11. The system for accessing a surgical target site of claim 1, wherein the lateral, trans-psoas path toward the target site has a longitudinal axis and a cross-sectional dimension, said cross-sectional dimension having a non-circular shape and comprises a non-radially symmetric path, said non-radially symmetric path adapted to allow for expansion of an operative corridor such that contact with one or more nerves is avoided.

12. A method for performing lateral based minimally invasive surgical procedures utilizing progressively larger asymmetric cannulas, comprising:
providing a dilator system comprising a plurality of sequential dilators deliverable along a lateral, trans-psoas path to a targeted spinal site to create an operative corridor;
inserting a pilot cannula adapted for sensing the proximity of nerves;
verifying that said pilot cannula is not within a predetermined distance to a nerve;
based on a said verifying step indicating that said pilot cannula is not within said predetermined distance to a nerve, introducing a first dilator having a first end, a second, and a longitudinal length therebetween, wherein the first dilator has at least one end that comprises an asymmetric cross-sectional shape;
introducing a second dilator about said first dilator, said second dilator having a first end, a second end, and a longitudinal length therebetween, wherein the second dilator comprises an asymmetric cross-sectional shape;
introducing a third dilator about said second dilator, said third dilator having a first end, a second end, and a longitudinal length therebetween, wherein the third dilator comprises an asymmetric cross-sectional shape;
introducing a working cannula deliverable along said lateral, trans-psoas path to a targeted spinal site to maintain an operative corridor;
securing said working cannula at least one of a distal end and a proximal end of said working cannula, whereby said method reduces the risk that nerves will be impinged by contact with a cannula or a dilator, thereby reducing the risk of injury to a patient.

13. The method of claim 12, wherein said method further comprises:
determining whether said working cannula requires an extension cannula based on indicia provided on said working cannula;
selecting an extension cannula based on said determining step; and
securing said extension cannula to said working cannula.

14. A system for performing spine surgery, comprising:
a surgical access instrument deliverable along a lateral, trans-psoas path to a targeted spinal site to create an operative corridor;
said surgical access instrument having a proximal portion, a distal portion, and an elongate length with a longitudinal axis therebetween;
said distal portion having a non-circular asymmetric cross sectional shape with a convex portion;
said distal portion having at least one nerve stimulation electrode located thereon; and
said convex portion being selectively and radially expandable from a first initial position.

15. The system for performing spine surgery of claim 14, wherein said distal portion comprises a piezoelectric material.

16. The system for performing spine surgery of claim 14, wherein said proximal portion is selectively and radially expandable from a first initial position.

17. The system for performing spine surgery of claim 14, wherein at least a portion of said elongate length comprises a rigid non-expandable material.

18. The system for performing spine surgery of claim 14, wherein said distal portion comprises a plurality of selectively movable hinged portions.

19. The system for performing spine surgery of claim 14, further comprising two or more dilators having a first end, a second, and a longitudinal length therebetween, said two or more dilators comprising an asymmetric cross-sectional shape.

20. The system for performing spine surgery of claim 18, wherein each of said plurality of selectively movable hinged portion comprises a nerve stimulation electrode adapted to detect potential nerve impingement.
